(19) United States
(12) Patent Application Publication
(10) Pub. No.: US 2024/0069027 A1
(43) Pub. Date: Feb. 29, 2024

(54) USE OF NITROGEN-DOPED CARBON FLUORESCENT QUANTUM DOT IN PREPARATION OF PRODUCT FOR DETECTING AEROBIC GLYCOLYSIS

(71) Applicants: SHANGHAI NINTH PEOPLE'S HOSPITAL, SHANGHAI JIAOTONG UNIVERSITY SCHOOL OF MEDICINE, SHANGHAI (CN); SHANGHAI INSTITUTE OF MICROSYSTEM AND INFORMATION TECHNOLOGY, CHINESE ACADEMY OF SCIENCES, Shanghai (CN)

(72) Inventors: JIPENG LI, SHANGHAI (CN); SIWEI YANG, SHANGHAI (CN); GUQIAO DING, SHANGHAI (CN); HUIFANG ZHOU, SHANGHAI (CN); XIANQUN FAN, SHANGHAI (CN)

(73) Assignees: SHANGHAI NINTH PEOPLE'S HOSPITAL, SHANGHAI JIAOTONG UNIVERSITY SCHOOL OF MEDICINE, SHANGHAI (CN); SHANGHAI INSTITUTE OF MICROSYSTEM AND INFORMATION TECHNOLOGY, CHINESE ACADEMY OF SCIENCES, (omitted)

(21) Appl. No.: 18/035,270

(22) PCT Filed: Dec. 28, 2021
(86) PCT No.: PCT/CN2021/141880

§ 371 Date: Nov. 13, 2023

(30) Foreign Application Priority Data
Nov. 5, 2020 (CN) 202011222226.8
Oct. 29, 2021 (CN) 202111275141.0

(51) Int. Cl. G01N 33/574; C01B 32/194; C09K 11/65; G01N 21/64

(52) U.S. Cl. CPC G01N 33/57407; C01B 32/194; C09K 11/65; G01N 21/6428; G01N 21/6458; G01N 33/57492; B82Y 20/00; C01B 2204/32; C01P 2002/54; C01P 2004/64; C01P 2006/60

(57) ABSTRACT

A use of nitrogen-doped carbon fluorescent quantum dots in preparation of aerobic glycolysis detection products is provided. The carbon-nitrogen fluorescent quantum dots are selected from one or more of C₃N₄ quantum dots, C₂N quantum dots, and C₃N quantum dots. The aerobic glycolysis detection products are reagents, based on a final volume of the reagents, the reagents comprise the carbon-nitrogen fluorescent quantum dots with a final concentration of 1 μg/mL-1 mg/mL. The present disclosure realizes fluorescent labeling of NAD⁺ in living cells using the carbon-nitrogen fluorescent quantum dots, thus achieving fluorescent labeling and imaging of cells having aerobic glycolysis, which has the advantages of low cost, high efficiency, rapidity, and high accuracy. Meanwhile, the present disclosure is conducive to developing a series of technologies such as fluorescence identification of exfoliated tumor cells, very early warning of tumors, detection of tumor metastases, and assessment of tumor proliferation and malignancy.

### FIELD OF THE INVENTION

The present disclosure relates to the field of aerobic glycolysis, in particular, to a use of nitrogen-doped carbon fluorescent quantum dots in preparation of aerobic glycolysis detection products.

### BACKGROUND OF THE INVENTION

The aerobic glycolysis process is an important metabolic process that differentiates normal cells and tumor cells, and therefore, fluorescence screening and imaging of aerobic glycolysis at the cellular level is a potentially important method to achieve tumor cell identification and tumor risk assessment. The detection of aerobic glycolysis is mainly dependent on the detection of NAD⁺ or NADH. With the development of technology, for the detection of specific target metabolites in glycolysis, the traditionally technical means include enzymatic cycling assay, chromatography, mass spectrometry, and nuclear magnetic resonance. The limitations of these assays are that they reflect the average metabolic state of a cell population, cannot reflect the metabolic state of a single cell, require cell lysates, and cannot be performed on live cells, much less in vivo. “Genetically encoded metabolite sensors” is a cutting-edge detection technology, which realizes labeling based on the principle that the fluorescent proteins change their structure and fluorescence intensity after binding to metabolites, such as NADH, ATP, glucose, and the like. See, SoNar, a Highly Responsive NAD⁺/NADH Sensor, Allows High-Throughput Metabolic Screening of Anti-tumor Agents. Genetically encoded metabolite sensors can sensitively reflect the dynamics change of intracellular NAD⁺ and NADH, but they also have limitations, such as weak fluorescence intensity, low specificity (whole-cell NAD⁺ detection), and high susceptibility to pH (tumor acidic microenvironment).

### SUMMARY OF THE INVENTION

In view of the above-mentioned drawbacks, the present disclosure provides a use of nitrogen-doped carbon fluorescent quantum dots in preparation of aerobic glycolysis detection products to solve the problems in the prior art.

The present disclosure also provides a method for detecting aerobic glycolysis, comprising the following steps: co-incubating an aerobic glycolysis detection product comprising a nitrogen-doped carbon fluorescent quantum dot with a sample to be tested, and detecting whether the sample to be tested is fluorescent or measuring a fluorescence intensity of the sample to be tested after the incubation.

The use of nitrogen-doped carbon fluorescent quantum dots in preparation of aerobic glycolysis detection products of the present disclosure has the following beneficial effects: it can realize fluorescent labeling of NAD⁺ in living cells using nitrogen-doped carbon fluorescent quantum dots, and then realize fluorescent labeling and imaging of cells with aerobic glycolysis, which has the advantages of low cost, high efficiency, rapidity, and high accuracy. Meanwhile, the present disclosure is conducive to developing a series of techniques such as fluorescence identification of exfoliated tumor cells, very early warning of tumors, detection of tumor metastases, and assessment of tumor proliferation and malignancy.

The conception, specific features, and resulting technical effects of the present disclosure will be further described in the following with reference to the accompanying drawings to ease the fully understanding of the purpose, features, and effects of the present disclosure.

### BRIEF DESCRIPTION OF DRAWINGS

FIG. 1 shows common cellular metabolites assayed by an aerobic glycolysis detection product of the present disclosure.

FIG. 2 shows fluorescence excitation wavelengths and fluorescence emission wavelengths of metabolite NAD⁺ and nitrogen-doped carbon fluorescent quantum dots of the present disclosure, where nitrogen-doped carbon fluorescent quantum dots are a major component of the aerobic glycolysis product.

FIG. 3 shows a fluorescence-concentration curve of metabolite NAD⁺ assayed by the aerobic glycolysis detection product of the present disclosure.

FIG. 4 shows aerobic glycolysis in A375 cells and fibroblasts assayed by the aerobic glycolysis detection product of the present disclosure.

FIG. 5 shows a fluorescence diagram of PFK15-treated A375 cells and untreated A375 cells assayed by the aerobic glycolysis detection product of the present disclosure.

FIG. 6 shows a fluorescence diagram of tumors in animals assayed by the aerobic glycolysis detection product of present disclosure.

FIG. 7 shows a fluorescence diagram of tumor cells in a urine sample assayed by the aerobic glycolysis detection product of the present disclosure.

### DETAILED DESCRIPTION OF THE INVENTION

The present disclosure will be described in detail by using the embodiments below.

The present disclosure provides a use of nitrogen-doped carbon fluorescent quantum dots in preparation of aerobic glycolysis detection products.

The carbon-nitrogen fluorescent quantum dots (i.e., nitrogen-doped carbon fluorescent quantum dots, N-CDs) are selected from one or more of C₃N₄ quantum dots, C₂N quantum dots, and C₃N quantum dots.

The N-CDs have a homogeneous dimension and their nitrogen-doped lattice structure significantly improves the fluorescence quantum efficiency of N-CDs, giving them strong and stable photoluminescence characteristics at 540 nm. In bioimaging, N-CDs exhibit obvious advantages, such as strong fluorescence signals, high detection sensitivity, good stability, good biocompatibility, and long-time dynamic observation and in vivo imaging, compared with traditional fluorescence imaging agents.

In an embodiment, the carbon-nitrogen fluorescent quantum dots are C₃N quantum dots. C₃N quantum dots are a single-layered two-dimensional semiconductor quantum material and have a honeycomb-shaped non-porous ordered structure, similar to that of graphene. C₃N quantum dots are composed of carbon and nitrogen atoms and are a novel indirect bandgap semiconductor material.

In one embodiment, an intrinsic bandgap of the C₃N quantum dots is 0.39 eV, and the bandgap can be adjusted based on the nano-size effect. The on/off ratio of field effect transistor (FET) devices based on single-layered C₃N films can be as high as 5.5×10¹⁰, and their carrier mobility can be as high as 220 cm2V⁻¹ s⁻¹. By adjusting the size of C₃N quantum dots, photoluminescence at about 400-900 nm can be achieved.

Electron injection can be realized by hydrogenation of C₃N quantum dots and C₃N quantum dots can produce long-range ferromagnetism at a temperature below 96 K. The bandgap of C₃N quantum dots makes up for the lack of an intrinsic bandgap of graphene, injection of hydrogenated carrier provides a new means for regulating the electrical properties of the material, and ferromagnetism indicates that the material system has rich physical connotation.

The content of Nitrogen (N) in the carbon-nitrogen fluorescent quantum dots is 0.5-5 at %. The content of N in the carbon-nitrogen fluorescent quantum dots may in any one of the following ranges: 0.5-1.5 at %, 1.5-2.5 at %, 2.5-3.5 at %, 3.5-4.5 at %, and 4.5-5 at %.

The diameter of each of the carbon-nitrogen fluorescent quantum dots is 1-100 nm. The diameter of the carbon-nitrogen fluorescent quantum dots may in any one of the following ranges: 1-10 nm, 10-20 nm, 20-30 nm, 30-40 nm, 40-50 nm, 50-60 nm, 60-70 nm, 70-80 nm, 80-90 nm, and 90-100 nm.

In one embodiment, the quantum yield of the carbon-nitrogen fluorescent quantum dots is in a range of 0.1-0.9.

In one embodiment, the excitation wavelength of the carbon-nitrogen fluorescent quantum dots is in a range of 240-650 nm, and/or, the emission wavelength of the carbon-nitrogen fluorescent quantum dots is in a range of 350-950 nm.

In one embodiment, there is no need to modify surfaces of the carbon-nitrogen fluorescent quantum dots.

In one embodiment, the use is in preparation of aerobic glycolysis detection products for living cells. In one embodiment, the use is in preparation of aerobic glycolysis detection products for single cells. Further, the living cells or single cells are living cells or single cells having an aerobic glycolysis metabolic mode.

Further, the living cells or single cells having an aerobic glycolysis metabolic mode are tumor living cells or tumor single cells. Fluorescence enhancement is achieved by fluorescence resonance energy transfer of the carbon-nitrogen fluorescent quantum dots and oxidized form of nicotinamide-adenine dinucleotide (NAD⁺) which is a metabolic intermediate product of cell aerobic glycolysis

The aerobic glycolysis detection product determines the aerobic glycolysis by detecting NAD⁺. That is, the use is in preparation of NAD⁺ detection products for living cells.

Further, the living cells or single cells are tumor living cells or tumor single cells generating NAD⁺.

The aerobic glycolysis detection product is a reagent, and based on the final volume of the reagent, the reagent includes the carbon-nitrogen fluorescent quantum dots with a final concentration ranging from 1 μg/mL to 1 mg/mL.

The reagent further includes a buffer, which serves as a solvent for the carbon-nitrogen fluorescent quantum dots, and the buffer is selected from one or more of physiological saline, water, dimethyl sulfoxide (DMSO), N,N-dimethylformamide (DMF), and poly (butylene succinate) (PBS). The pH of PBS is 7.2-7.4.

The present disclosure further provides a use of nitrogen-doped carbon fluorescent quantum dots in preparation of NAD⁺ detection products.

The present further provides an aerobic glycolysis detection product, comprising the carbon-nitrogen fluorescent quantum dots.

The carbon-nitrogen fluorescent quantum dots are selected from one or more of C₃N₄ quantum dots, C₂N quantum dots, and C₃N quantum dots.

The aerobic glycolysis detection product also includes a buffer. The buffer serves as a solvent for the carbon-nitrogen fluorescent quantum dots, and the buffer is selected from one or more of physiological saline, water, dimethyl sulfoxide (DMSO), N,N-dimethylformamide (DMF), and poly (butylene succinate) (PBS).

The present disclosure further provides a use of nitrogen-doped carbon fluorescent quantum dots in preparation of product for detecting or treating tumor.

The tumor detection product is used for early diagnosis of tumors. Specifically, the tumor detection product is used for tumor cell identification, tumor small-lesion detection, tumor cell screening in clinical samples, or tumor visualization research.

The tumor detection product is used for early intervention of tumor.

The tumor cell detection product at least comprises the carbon-nitrogen fluorescent quantum dots.

The present disclosure also provides a method for detecting aerobic glycolysis, comprising the following steps: co-incubating an aerobic glycolysis detection product comprising nitrogen-doped carbon fluorescent quantum dots with a sample to be tested, and detecting whether the sample to be tested is fluorescent or measuring a fluorescence intensity of the sample to be tested after the incubation.

In one embodiment, the method further comprises the following steps: centrifuging after incubation, discarding the supernatant, resuspending the precipitate with a buffer, and detecting whether there is fluorescence or any detectable fluorescence intensity after the resuspending.

The method also includes one or more of the following features:
1) the sample to be tested is a sample of living cells having aerobic glycolysis metabolic characteristics; preferably, the sample to be tested is selected from cells, tumor tissues or non-tumor tissues, hydrothorax, blood or urine; more preferably, the sample to be tested is a sample after pre-treatment;
2) the volume of the aerobic glycolysis detection product to be used is in a range of 1 μL˜1 mL;
3) the co-incubation time is in a range of 5 min˜2 h;
4) the co-incubation temperature is in a range of 4˜50° C.; preferably, the co-incubation temperature is in a range of 20˜40° C.;
5) when detecting fluorescence intensity, the excitation wavelength of fluorescence to be detected is in a range of 200˜800 nm;
6) the centrifugation speed is in a range of 500˜1500 rpm/min, and the centrifugation time is in a range of 1-30 minutes. In one embodiment, the pre-treatment of the sample to be tested comprises the following:
step 1: shearing the collected tissue to be tested into 1 mm³ volume for culture, or centrifuging the collected cells, hydrothorax, blood or urine and discarding the supernatant;
step 2: adding the buffer, resuspending the tissue fragment or sediment obtained in step 1, and mixing and incubating the resulting resuspension with the aerobic glycolysis detection product.

In an embodiment, the buffer used in step 2 in the pre-treatment of the sample to be tested is the same as the buffer used in the aerobic glycolysis detection product.

In one embodiment, a fluorescence microscope is used to detect whether there is fluorescence. Cells having aerobic glycolysis can emit fluorescence.

A fluorescence spectrophotometer is used to obtain the fluorescence intensity. Semi-quantitative analysis of the sample is performed based on the obtained fluorescence intensity.

In one embodiment, the excitation wavelength of the fluorescence microscope is in a range of 200-800 nm.

Generally, methods for detecting aerobic glycolysis include ones for diagnostic purposes and ones for non-diagnostic purposes. Preferably, the methods for detecting aerobic glycolysis are for non-diagnostic purposes. The non-diagnostic purposes may include the detection of aerobic glycolysis in scientific research to study the mechanism of aerobic glycolysis, the mechanism of disease development, the metabolic mechanism of cells, etc.

Embodiments of the present disclosure are illustrated below by specific examples, and other advantages and efficacy of the present disclosure can be readily understood by those skilled in the art according to the contents in this specification. The present disclosure may also be implemented or applied by different specific embodiments, and the details in this specification may be modified or changed in various ways without departing from the spirit of the present disclosure based on different views and applications.

Before further describing specific embodiments of the present disclosure, it should be understood that the protection scope of the present disclosure is not limited to the specific embodiments described below. It should also be understood that the terms used in embodiments of the present disclosure are intended to describe specific embodiments and are not intended to limit the protection scope of the present disclosure. In the specification and the claims of the present disclosure, unless otherwise stated, the singular forms “a”, “one”, and “this” include the plural form.

When a numerical range is given in an embodiment, it should be understood that, unless otherwise stated, the two endpoints of each range of any value between the two endpoints may be chosen. Unless otherwise defined, all technical and scientific terms used in the present disclosure have the same meaning as commonly understood by those skilled in the art. In addition to the specific method, apparatus, and material used in the embodiments, any method, apparatus, and material of the prior art similar to or equivalent to the method, apparatus, and material described in the embodiments of the present disclosure may be used to implement the present disclosure according to the knowledge of the prior art by a person skilled in the art and the contents of the present invention.

The preparation method of C₃N quantum dots used in the following embodiments is as follows: 80 mL of 2,3-diaminophenazine solution (2.0 mM) was added into a 100 mL autoclave, heated, and maintained at 380° C. for 16 h to obtain C₃N quantum dots, and then the product was filtered by an aluminum oxide membrane with a pore size of 0.02 μm, the resulting filtrate was stood for 12 hours to obtain C₃N quantum dots that can be directly used in biological experiments. The 2,3-diaminophenothiazine (DAP, 98%) was purchased from US J&K Chemical Technologies, Inc.

### Embodiment 1

The solutions of various cellular metabolic intermediates (0.9% saline) were selected as the samples to be tested, and the intermediates were glucose, PKM1, PKM2, Pyr, LDH, Lactate, NADH, NAD⁺, ADP, O₂₋, ·OH, etc. (all with a concentration of 0.1 mM). C₃N quantum dots (with the diameter of 1 nm, the concentration of 1 μg/mL, the solvent of saline) were selected as the aerobic glycolysis detection product. C₃N quantum dots were added to the samples to be tested and incubated for 2 h at 25° C. The excitation wavelength of the fluorescence spectrophotometer was set to 400 nm, and the fluorescence intensity was read.

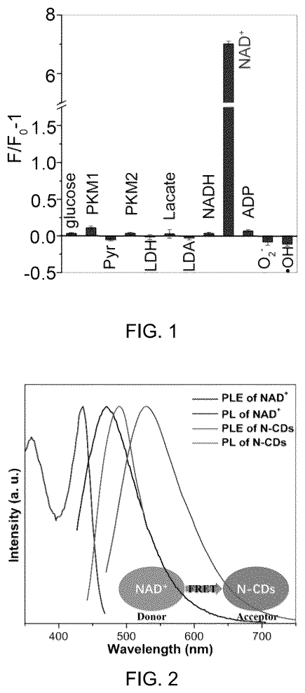

The results were shown in FIG. 1: the fluorescence intensity of NAD⁺ samples increased by about 7 times, and the fluorescence intensity of the remaining samples did not change significantly. Subsequently, a NAD⁺ solution (with a concentration of 0.1 mM) and a C₃N quantum dots solution (1 μg/mL) were selected as samples to be tested, and the fluorescence excitation spectra and fluorescence emission spectra of the two solutions were obtained, respectively. As shown in FIG. 2, the excitation wavelength and emission wavelength of the NAD⁺ solution were 423 nm and 476 nm, respectively, and the excitation wavelength and emission wavelength of the C₃N quantum dots solution were 495 nm and 530 nm, respectively. Therefore, it can be seen that in the process of fluorescence resonance energy transfer, NAD⁺ was energy donors, and C₃N quantum dots were energy acceptors.

The above results indicate that fluorescence resonance energy transfer was realized by C₃N quantum dots and NAD⁺, with NAD⁺ being a metabolic intermediate product of cell aerobic glycolysis.

### Embodiment 2

NAD⁺ solutions were selected as the samples to be tested, and the concentrations were 0 (i.e., physiological saline), 0.05, 0.1, 0.15, 0.2, and 0.25, respectively.

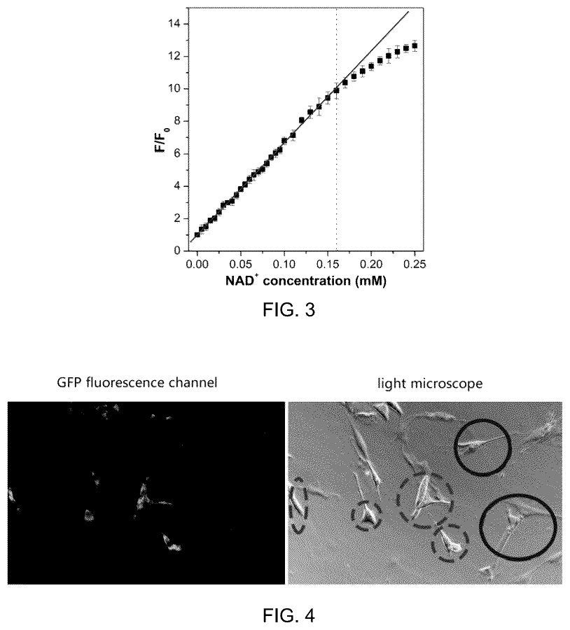

C₃N quantum dots (with the diameter of 1 nm, the concentration of 1 μg/mL, the solvent of saline) were selected as the aerobic glycolysis detection product. C₃N quantum dots were added to the samples to be tested and incubated for 2 h at 25° C. The fluorescence intensity was collected by fluorescence spectrophotometer. The results were shown in FIG. 3, it can be seen that the fluorescence intensity of C3N quantum dots increases with the increment of NAD⁺ concentration within a certain range.

### Embodiment 3

A375 cells (which have aerobic glycolysis metabolic mode) and fibroblasts (oxidative phosphorylation metabolic mode) were selected as the samples to be tested, and the two types of cells were co-cultured with a concentration of 103. C₃N quantum dots (with a diameter of 1 nm, a concentration of 1 μg/mL, a solvent of saline) were selected as the aerobic glycolysis detection product. 1 μL of C₃N quantum dots were added to the samples to be tested and incubated for 2 h at 25° C. The fluorescence of the samples was observed by a fluorescence microscope (with an excitation wavelength of 400 nm). The results were shown in FIG. 4: A375 cells emitted clearly visible fluorescence in the field of view, while fibroblasts did not emit visible fluorescence. The dashed circles represent A375 cells, and the solid circles represent fibroblasts.

### Embodiment 4

A375 cells were selected as the samples to be tested with a cell concentration of 10⁵.

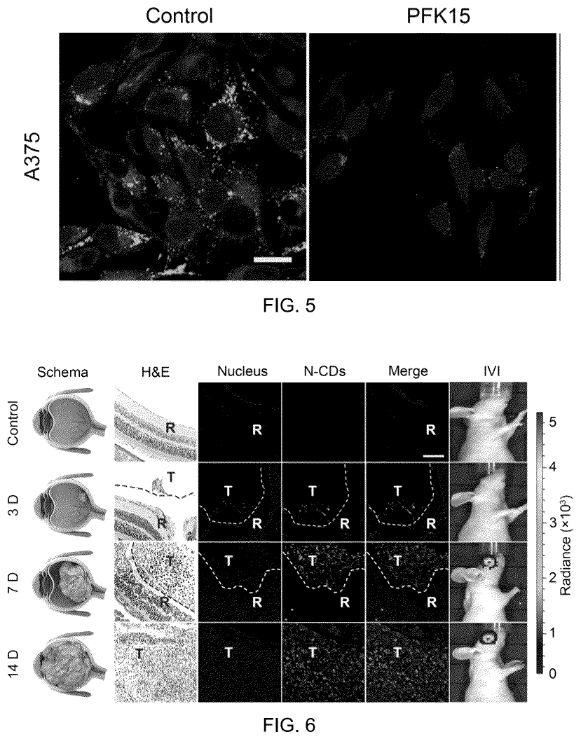

1-(4-pyridinyl)-3-(2-quinolinyl)-2-propen-1-one (PFK15, 20 nM) was added to the cell samples to be tested and incubated for 12 h. C₃N quantum dots (with a diameter of 1 nm, a concentration of 1 μg/mL, a solvent of saline) were selected as the aerobic glycolysis detection product. 1 μL of C₃N quantum dots were added to the samples to be tested and incubated for 2 h at 25° C. The fluorescence of the samples was observed by a fluorescence microscope (with an excitation wavelength of 400 nm). It can be seen that the fluorescence of the A375 cells with PFK15 treatment (PFK15 can block the aerobic glycolysis metabolism and reduce the NAD⁺ concentration in cytoplasm) was decreased by 43% compared with that of the group without PFK15 treatment (results were shown in FIG. 5).

### Embodiment 5

BALB/c nude mice (4 weeks, and female) were anesthetized with 3% pentobarbitone. A375 cells were added to a suspension with a concentration of 2×10⁸ mL⁻¹.

Tumor cells were injected into vitreous cavities of mice to establish an intraocular orthotopic tumor animal model. One week after the injection of the tumor cells, a C₃N quantum dots solution was injected into the vitreous cavities and incubated for 12 h. Small animal in vivo imaging was then performed, followed by execution of the mice, and the eye tissues were sectioned for HE staining and fluorescence photography. As shown in FIG. 6, tumor cell growth was visible in HE staining, fluorescence photography, and small animal in vivo imaging. The results indicate that carbon-nitrogen fluorescent quantum dots accurately realize dynamic fluorescence labeling and monitoring of the tumor growth process of cells having the aerobic glycolysis metabolic mode in vivo.

### Embodiment 6

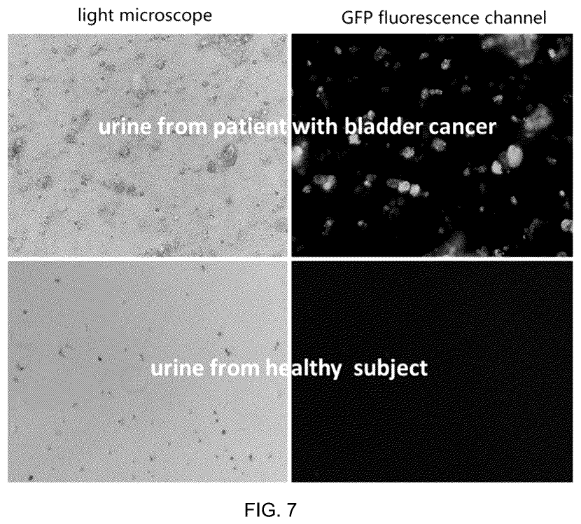

Urine from patients with bladder cancer and healthy subjects was selected as the samples to be tested. C₃N quantum dots (with the diameter of 1 nm, the concentration of 1 μg/mL, the solvent of saline) were selected as the aerobic glycolysis detection product. 1 μL of C₃N quantum dots were added to the samples to be tested and incubated for 2 h at 25° C. The fluorescence of the samples was observed by a fluorescence microscope (with an excitation wavelength 400 nm). The results were shown in FIG. 7: urine cells from patients with cancer emitted clearly visible fluorescence in the field of view, while urine cells from healthy subjects did not emit visible fluorescence.

The above embodiments are intended to illustrate the disclosed embodiments of the present disclosure and shall not to be construed as a limitation of the present disclosure. Furthermore, the various modifications listed herein and variations of the method in the present disclosure are apparent to those skilled in the art without departing from the scope and spirit of the present disclosure. Although the present disclosure has been described specifically by a variety of specific preferred embodiments of the present disclosure, it should be understood that the present disclosure is not limited to these specific embodiments. In fact, various modifications as described above that would be obvious to those skilled in the art should be included within the scope of the present disclosure.

1. A use of nitrogen-doped carbon fluorescent quantum dots in preparation of aerobic glycolysis detection products.

2. The use according to claim 1, wherein the nitrogen-doped carbon fluorescent quantum dots are selected from one or more of N-doped graphene quantum dots, C₃N₄ quantum dots, C₂N quantum dots, and C₃N quantum dots.

3. The use according to claim 1, wherein a nitrogen content of the nitrogen-doped carbon fluorescent quantum dots is in a range of 0.5-5 at %.

4. The use according to claim 1, wherein a diameter of the nitrogen-doped carbon fluorescent quantum dots is in a range of 1-100 nm.

5. The use according to claim 1, wherein the use is in preparation of aerobic glycolysis detection products for living cells, preferably, the use is in preparation of quantitative or semi-quantitative aerobic glycolysis detection products.

6. The use according to claim 1, wherein an NAD⁺ detection of the aerobic glycolysis detection products is used to determine the aerobic glycolysis.

7. The use according to claim 1, wherein the aerobic glycolysis detection products are reagents, and the reagents, based on a final volume of the reagents, comprise the nitrogen-doped carbon fluorescent quantum dots with a final concentration ranging from 1 μg/mL to 1 mg/mL.

8. The use according to claim 7, wherein the reagents further comprise a buffer, and the buffer is selected from one or more of saline, water, DMSO, DMF, and PBS.

9. A use of nitrogen-doped carbon fluorescent quantum dots in preparation of NAD⁺ detection products.

10. A method for detecting aerobic glycolysis, comprising the steps of: co-incubating an aerobic glycolysis detection product comprising a nitrogen-doped carbon fluorescent quantum dots with a sample to be tested, and detecting whether the sample to be tested is fluorescent or measuring a fluorescence intensity of the sample to be tested after the incubation.

11. The method according to claim 10, comprising the steps of: centrifuging after the incubation, discarding a supernatant, resuspending a precipitate with a buffer, and then detecting whether the sample to be tested is fluorescent or measuring a fluorescence intensity of the sample to be tested.

12. The method according to claim 10, further comprising one or more of the following features:
1) the sample to be tested is a sample of living cells having aerobic glycolysis metabolic characteristics; preferably, the sample to be tested is selected from cells, tumor tissue or non-tumor tissue, hydrothorax, blood or urine; more preferably, the sample to be tested is a sample after pre-treatment;
2) a volume of the aerobic glycolysis detection product to be used is in a range of 1 μL˜1 mL;
3) a co-incubation time is in a range of 5 min˜2 h; and
4) a co-incubation temperature is in a range of 4˜50° C.;
wherein when detecting fluorescence intensity, an excitation wavelength of fluorescence to be detected is in a range of 200˜800 nm.
